(19) United States
(12) Patent Application Publication
(10) Pub. No.: US 2021/0371788 A1
(43) Pub. Date: Dec. 2, 2021

(54) APPARATUS AND METHOD FOR CULTURING CELLS

(71) Applicant: CESCO BIOENGINEERING CO., LTD., Taichung (TW)

(72) Inventors: Mao-Yen HUANG, TAICHUNG (TW); King-Ming CHANG, TAICHUNG (TW)

(21) Appl. No.: 17/325,499

(22) Filed: May 20, 2021

Related U.S. Application Data
(60) Provisional application No. 63/030,278, filed on May 26, 2020.

(51) Int. Cl. C12M 1/04; C12M 1/00; C12M 1/12; C12M 3/04

(52) U.S. Cl. CPC C12M 23/24; C12M 23/38; C12M 27/10; C12M 23/50; C12M 25/14

(57) ABSTRACT

An apparatus and method for preparing and culturing cells comprises a gas permeable chamber having at least an inlet and an outlet for introducing culture medium and cells. A cell growth substrate placed at the end(s) of the gas permeable chamber for cells anchored or embedded. The present invention discloses a simple but efficient cell culture method by slowly rotating the cell culture apparatus horizontally combining with a partially filled culture media in the gas permeable container in order to be able to expose the carriers and submerge the carriers intermittently, to achieve an optimal cell culture environment through the efficient oxygen transfer, CO2 balance, with sufficient nutrient supply, without vigorous agitation to achieve oxygen transfer. The horizontally rotation movement with the present cell growth apparatus efficient the nutrition, carbon dioxide and oxygen transfer during cultured process for production of cellular protein products, viruses or harvesting the cells.

### CROSS-REFERENCE TO RELATED APPLICATIONS

This is a non-provisional application which claims benefit of a Provisional Patent Application No. 63/030,278 filed May 26, 2020, the disclosure of which is hereby incorporated by references.

### BACKGROUND OF THE INVENTION

### 1. Field of the Invention

The present invention relates to an apparatus and a method for growing the animal cells and/or tissue culture in vitro. More particularly, the present invention relates to a cell culture apparatus with a gas permeable container and a cell culture method using the cell culture apparatus.

### 2. Description of the Prior Art

Cell culture processes have been developed extensively over the years for the growth of bacteria, yeast and molds, all of which typically possess robust cell walls and/or extra cellular materials thus, are more resilient. The structural resilience of these microbial cells is a key factor contributing to the rapidity of the development of high-efficient cell culture processes for these types of cells. For example, most bacterial cells can be grown in very large volumes of liquid medium using vigorous agitation, culture stirring and gas sparging techniques to achieve good aeration during growth, all the while maintaining viable cultures.

In contrast, the techniques to culture cells such as eukaryotic cells, animal cells, mammalian cells and/or tissue are more difficult and complex since these cells are more delicate than microbial cells and have nutrient and oxygen requirements during growth which are more complex and difficult to maintain. Further, animal cells and/or mammalian cells cannot withstand the excessive turbulence and/or shear forces that can be created by an influx of air or gaseous mixtures, such as a mixture containing oxygen, nitrogen and carbon dioxide, that are tolerated more easily by microbial cells. In addition, no animal cells can be directly exposed to gases. Most of the animal cells can only utilize dissolved oxygen in the culture medium. Animal cells and mammalian cells are more likely to be damaged by air and gas influx than are microbial cells and thus, results in increased cell mortality. Cell culture devices for cell culturing often have internal moving parts, such as an impeller, which subject the cells to a very high fluid shearing force causing cell damage, sometimes cell death, thus leading to low viability of cultures and as a result reduces protein and/or cell by-product production. Likewise, cell culture devices that utilize other types of mechanical parts, harsh air movement, or abrupt fluid movement as a mechanism to achieve cell suspension and/or proper aeration will likely cause damage to cells and hinder cell and tissue growth, which further leads to a decrease in cell by-product production, such as viruses, antibodies, and protein.

A primary function of a cell culture device is for research wherein large numbers of cells are grown to refine the minute quantities of an active material, including but not limited to a protein or antibody that are secreted by cells into the growth medium. Because of the need to culture eukaryotic cells and/or prokaryotic cells and/or animal cells and/or mammalian cells in the laboratory, cell culture devices and culturing apparatuses have become an important tool in research and in the production of cells for producing active proteins and/or antibodies and/or any cell by-products.

Many methods are known in the art for growing cells in culture, both on small and large scales. For smaller-scale cell culturing, many vessels have been developed over the years. Cell culture dishes, for example, represent one type of culturing vessel. Cell culture petri dishes typically consist of a bottom dish, which contains the growth medium, and a removable cover. Although the removable cover provides a convenient access to the culture, microorganisms as a result of repeating removing the cover during the culturing process often and easily contaminate cells. In fact, contamination is one of the principal challenges to successful cell and tissue culturing techniques.

To overcome contamination with culture dishes, culture flasks were developed. Culture flasks typically have a culture chamber, a small tubular opening located at one end of the flask and a corresponding closure. This design attempts to minimize the exposure of cells to dust, bacteria, yeast and other contaminants. For example, patents teaching culture flasks can be found in U.S. Pat. No. 4,334,028 to Carver and U.S. Pat. No. 4,851,351 to Akamine and U.S. Pat. No. 5,398,837 to Degrassi. Although culture flasks were an improvement over culture dishes, they did not fully remedy the contamination problem. In addition, neither the culture dish nor the culture flask can provide appropriate aeration to cells. Furthermore, the growth surface area available in culture flasks is not adequate, as in culture dishes; thus, placing limits on scaling-up the culturing process using this technology.

Another technology developed for use in cell and tissue culturing were roller bottles. The roller bottle has been widely used in the art for many years. Although they offer some advantages over dishes and flasks, such as a larger surface area for cell attachment and growth, they are still unable to remedy all of the deficiencies and particularly with scaling up. Examples of patents directed to roller bottles include U.S. Pat. No. 5,527,705 to Mussi et al. and U.S. Pat. No. 4,962,033 to Serkes.

Moreover, although the surface area of roller bottles is greater by comparison to culture flasks and dishes, it is often not considered adequate since the surface for cell adhesion is not necessarily more favorable than the culture flasks and dishes, particularly for scaling up the growth of cell cultures. Some efforts have been made to improve upon the roller bottle by providing a greater amount of surface area per roller bottle. For example, U.S. Pat. No. 5,010,013 to Serkes describes a roller bottle with increased surface area for cell attachment. Serkes relates to the use of corrugated channels added to the interior surface area of the roller bottle to increase capacity for cellular attachment. However, a typical roller bottle provides only a surface area of about 850-1700 cm² for cultivating cells, a multitude of roller bottles are still required for scaling up production. Although, automation of culturing with a large plurality of roller bottles can save on time and labor investment, these operations are typically costly and limiting. Above cell culture devices, i.e. petri dishes, tissue culture flasks, and roller bottles, even very simple to use, all utilize the surface on the device itself and only a portion of culture medium can be filled in the device for oxygen transfer. Thus, the poor space utilization and it also requires intensive labors for operation. A simple but space saving cell culture device for laboratory or larger scale cell culture is still demanded in this application area.

In addition to the problems of hydrodynamic shear forces and surface-area limitations, a central problem inherent in cell and tissue culturing techniques is attaining and maintaining sufficient oxygenation in the growing culture. It is well-known in the art that prokaryotic cells, eukaryotic cells, including animal cells, mammalian cells, insect cells, yeast and molds all have one major rate-limiting step, oxygen mass transfer.

Oxygen metabolism is essential for metabolic function of most prokaryotic cells and eukaryotic cells with the exception of some fermentative-type metabolisms of various eukaryotic microorganisms, such as yeast. Particularly, with mammalian and animal cell culturing techniques, oxygen flux is especially important during the early stages of rapid cell division. Oxygen utilization per cell is greatest when cells are suspended; requirements for oxygen decrease as the cells aggregate and differentiate. Some mammalian and animal cells are anchorage-dependent, requiring a surface to grow, whereas other mammalian and animal cells are anchorage independent and can be grown in liquid environments regardless of the types of cells. However, these cells all require dissolved oxygen in the medium. Nevertheless, during the later phases of cell culture with both anchorage-dependent and independent cells, as the number of cells per unit volume increases, the bulk oxygen mass transfer requirement once again increases.

Traditionally, at least with anchorage-independent cells, increased requirements for oxygen are accommodated by mechanical stirring methods and the sparging of gases into the culture. However, as discussed, both stirring and the sparging of gases can result in damaging cells, thereby decreasing the viability of the culture and the overall efficiency and productivity of the cell and/or tissue culture. Further, direct sparging of cell and tissue cultures with gas can lead to foam production which, is also detrimental to cell viability.

Some attempts have been made in the art to solve the oxygenation problem during cell culturing. For example, U.S. Pat. No. 5,153,131, issued to Wolf et al. (“Wolf”), relates to a cell culture device vessel without mixing blades. Instead, air travels through an air inlet passageway through a support plate member across a screen and through a flat disk permeable membrane wedged between the two sides of the vessel housing. The oxygen then diffuses across the membrane into the culture chamber due to the concentration gradient between the two sides of the housing.

The Wolf cell culture device, however, presents many disadvantages. Particularly, the rate at which oxygen can diffuse across the disk-shaped membrane is a significant limitation that restricts the size of the culture chamber. Another disadvantage of the flat disk membrane is that it is designed to flex in order to cause mixing within the culture chamber, which can result in cell death. The mixing effect is a feature described as being critical for the distribution of air throughout the culture media, however, it will also tend to create shear forces within the chamber, again can be detrimental to cells, consequently providing sufficient gas exchange to sustain the growth of larger cellular structures is a significant and realistic restriction when designing a cell culture device or culture vessel.

An example showing an attempt to overcome the deficiencies thus far described is to make cell culture devices from gas permeable materials. For instance, U.S. Pat. No. 5,702,941, issued to Schwarz et al. (“Schwarz”), entitled “Gas Permeable Cell culture device And Method Of Use” relates to a vessel that is horizontally rotated and the vessel is at least partially composed of gas permeable materials. The gas exchange with the culture medium is intended to occur directly through the gas permeable materials of which the vessel walls are composed.

However, Schwarz discloses that the range of sizes for the vessel is still limited since gas exchange is dependent on the quantity of gas permeable surface area. Schwarz emphasized that as the surface area of the vessel increases, the volume and the amount of culture medium also increases. As such, the preferred dimensions of the vessel described in Schwarz are limited to between one and six inches in diameter while the width is, according to Schwarz, preferably limited to between one-quarter of one inch and one inch. Such size limitations are not suitable for growing three-dimensional cellular aggregates and tissues and/or any scaling up production.

Similarly, U.S. Pat. No. 5,449,617, issued to Falkenberg et al. (“Falkenberg”), entitled “Culture Vessel For Cell Culture” relates to a vessel that is horizontally rotated. The vessel is divided by a dialysis membrane into a cell culturing chamber and a nutrient medium reservoir. Gas permeable materials are used in the vessel walls to enable gas exchange in the cell culturing chamber. The Falkenberg vessel, however, is not designed to minimize turbulence within the cell culture chamber but rather, mixing is recited to be an essential step to keep the dialysis membrane wetted. Further, Falkenberg does not contemplate using the vessel to grow cellular aggregates or tissues of any kind.

Another solution has been to develop flexible, disposable plastic vessels that do not require cleaning or sterilization and require only minimal validation efforts. For example, U.S. Pat. No. 5,523,228 describes a flexible, disposable, and gas permeable cell culture chamber that is horizontally rotated. The cell culture chamber is made of two sheets of plastic fused together. The edges of the chamber, beyond the seams, serve as points of attachment to a horizontally rotating drive means. The cell culture chamber is made of gas permeable material and is mounted on a horizontally rotating disk drive that will support the flexible cell culture chamber without blocking airflow over the membrane surfaces. Thus, the cell culture chamber is placed in an incubator and oxygen transfer controlled by controlling the gas pressure in the incubator according to the permeability coefficient of the bag. The rotation of the bag assists in mixing the contents of the bag and enhances gas transfer throughout the bag. However, the cell culture chamber has no support apparatus and the aeration surface area cannot be proportional to volume when scale up, and it is therefore limited to small volumes. Furthermore, the cell culture chamber is flexible and is difficult to operate as a laboratory tool. Oxygen transfer through gas permeable film will build an oxygen layer between the film and culture media, which is toxic. A boundary will also form to restrict oxygen transfer if no mixing or poor mixing during culture. Those disadvantages all pose in the system where it is fill fully with culture medium without any gaseous headspace.

There is a continuing need to develop lightweight, pre-sterilized, disposable cell culture devices with simple connections to existing equipment. A member only requires little training to operate it, and it provides the necessary gas transfer and nutrient mixing required for successful cell cultures.

Given the importance of cell and tissue culture technology in biotechnology research, pharmaceutical research, patient care, academic research and in view of the deficiencies, obstacles and limitations exist in the prior art described, the present invention overcomes the obstacle and remedies the deficiencies in the prior art by teaching and disclosing a method and an apparatus for cell and tissue culturing that fulfills the long-felt need for a novel method and apparatus to culture cells and tissues that is more reliable, less complex, more efficient, less cumbersome, less expensive, less-labor intensive, eliminate oxygen boundary between gas permeable film layer and culture media, capable to grow 3D cells in high density without the limit of oxygen supply, capable of increasing cell vitality and producing a higher yield of cellular by-products generated from the cells.

### SUMMARY OF THE INVENTION

There are several obstacles remain unsolved in the cell culture technology. One of the greatest obstacles in cell and/or tissue culturing is that the apparatuses and/or method employed are difficult to strike a balance between providing enough oxygen but still can avoid injuring the cells. Another obstacle in mammalian cell culture is that a relatively large amount of inoculum is required to initiate a culture.

The invention comprehends both the apparatus and the method for use it. The apparatus for cell culturing having features of the present invention includes a semi-rigid gas permeable chamber which contains an inlet and/or an outlet, or a cap, and at least a cell growth substrate placing at the one end or both ends in the gas permeable culture chamber. The semi-rigid gas permeable chamber is made by silicone rubber with enough thickness, preferably 0.5 to 2 mm, that could stand still by itself without requiring extra supports. For laboratory use, the inlet and outlet ports are replaced with a cap that is more convenient for laboratory operation. For more secure and contamination-free operation, at least an inlet and an outlet ports are designed instead of cap. There is no air filter is needed due to the container itself is already gas permeable that allows oxygen and CO2 transfer and balance. The cell growth substrate, or called cell culture matrices or cell culture carriers, are placed at one end or two ends of the gas permeable chamber. The cell culture substrate is porous, and can be a sheet, multiple sheets or a plurality of discs disposed in a basket mounted at one end or two ends of the gas permeable chamber.

For cell culture, the chamber is secured on a horizontally rotated clamp, or platform driven by a step motor; a power connecting the motor to move the clamp or platform in a clockwise and/or counterclockwise direction; a timing controller controls the platform to stop at one end and keep for a period of time before turning back or keep turning to the other end. The movement could be clockwise all the time but stop at top and bottom end position for a set period of time; the movement could also be clockwise and stop on one end for a set period of time, then turning counterclockwise and stop on the other end for a set period of time.

The method for cell culturing in the present invention includes the steps of preparing a sterile gas permeable chamber which has a single hollow interior volume, mounting a cell growth substrate at one end or both ends in the chamber, introducing cell culture medium through a cap, or an inlet by partially fill the gas permeable chamber, at most be able to expose the cell growth substrate completely during rotation, placing cells suspension into the chamber to distribute cells on the cell growth substrate, securing the chamber on the platform, connecting a power to drive the motor and move the platform and invented device in rotating motion, setting up a timing control in order to keep one of cell culture substrate be submerged or exposed for a period of time in order to allow cells to anchor on the cell growth substrate or be embedded in the cell growth substrate, keeping the clockwise/counterclockwise motion until cells are anchored on growth substrate and/or embedded the cell growth substrate, setting up a timing control keeping one of cell culture substrate be submerged or exposed for a period of time in order to complete the nutrient exchange or control nutrient supply for both gaseous and liquid phase, keeping moving the platform in clockwise/counterclockwise motion wherein the cell culture substrate being submerged at one end, and then exposed after rotating to the other end, periodically repeating above steps from setting up timing control to the end process for maintaining suitable continual cell culture condition during the cultured period.

The apparatus and the method in the present invention providing a novel method for the culturing of cells in resolving the greatest obstacles about effectively oxygen/nutrient transfer, minimal metabolite waste accumulation, air bubbles and/or shear forces caused by an infusion of gases, culturing maximizes cells adhesion, increased surface area for air-medium contact and functions as a static mixer when the medium in the apparatus of the present invention.

The present invention provides a reliable, simple, inexpensive and efficient method for culturing cells and/or tissues and for harvesting cellular products produced thereof, such as prokaryotic cells, eukaryotic cells, animal cells, mammalian cells, whereby a continuous supply of both oxygen and nutrients to the cells are provided. Moreover, the method of the present invention reduces waste accumulation by providing sufficient oxygen during culture, helps removing excess carbon dioxide during culture, helps stabilizing culture environment with a simple, reliable, inexpensive and efficient means, helps preventing detrimental effects on cells caused by air bubbles and gases. Moreover, the method of the present invention could reduce initial seeding density that are usually required in animal cell culture, and also eliminate lag phase during initial growth period originally due to low inoculum density. Moreover, the present invention teaches and discloses a novel method for efficiently removing carbon dioxide and stabilizing pH during culture. Furthermore, the present invention provides a method for an easier and more convenient way to produce and harvest secreted cellular products, such as protein, and/or antibiotics, and/or any cellular and/or tissue products from cell or tissue cultures.

Accordingly, a cell culture apparatus includes a gas permeable container and at least one cell growth substrate which allows rapid and uniform transfer of gases between the environment of cells contained in the cell culture apparatus and the atmosphere of the incubator in which the cell culture apparatus is incubated.

Accordingly, a cell culture method is provided by adapting the cell culture apparatus for 3D cell culture with higher cell culture density. A simple but efficient cell culture method is provided by slowly rotating the cell culture apparatus horizontally combining with a partially filled culture media in the gas permeable container in order to achieve highly efficient oxygen transfer rate and carbon dioxide balance rate during cell culture.

Below, embodiments accompanied with the attached drawings are employed to explain the objectives, technical contents, characteristics and accomplishments of the present invention.

### BRIEF DESCRIPTION OF THE DRAWINGS

FIG. 1A and FIG. 1B show side views of a cell-cultivation apparatus of the first embodiment of present invention with pre-assemble and post-assemble.

FIG. 2A and FIG. 2B show side views of a cell-cultivation apparatus of the second embodiment of present invention with pre-assemble and post-assemble.

FIG. 3A and FIG. 3B show side views of a cell-cultivation apparatus of the third embodiment of present invention with pre-assemble and post-assemble.

FIG. 4A and FIG. 4B show side views of a cell-cultivation apparatus of the fourth embodiment of present invention with pre-assemble and post-assemble.

FIG. 5A and FIG. 5B show side views of a cell-cultivation apparatus of the fifth embodiment of present invention with pre-assemble and post-assemble.

FIG. 6A and FIG. 6B show side views of a cell-cultivation apparatus of the sixth embodiment of present invention with pre-assemble and post-assemble.

FIG. 7A and FIG. 7B show side views of a cell-cultivation apparatus of the seventh embodiment of present invention with pre-assemble and post-assemble.

FIG. 8 shows a side view of a cell-cultivation apparatus of the eighth embodiment of present invention.

FIG. 9A and FIG. 9B are schematically stereoscopic diagrams showing a device of the first and the second embodiments of present invention for driving the cell-cultivation apparatus.

FIG. 10A, FIG. 10B and FIG. 10C are schematically stereoscopic diagrams showing how the device rotates the cell-cultivation apparatus.

FIG. 11 is schematically stereoscopic diagram showing the device of the third embodiment of present invention for driving the cell-cultivation apparatus.

FIG. 12A, FIG. 12B and FIG. 12C are schematically side-view diagrams illustrating a cell-cultivation apparatus of gas permeability during rotating according to the present invention.

FIG. 13 shows a side view of a cell-cultivation apparatus of the nineth embodiment of present invention.

FIG. 14A and FIG. 14B show side views of a cell-cultivation apparatus of the tenth embodiment of present invention with pre-assemble and post-assemble.

### DESCRIPTION OF THE PREFERRED EMBODIMENTS

In according with the present invention, there is provided an apparatus and a method for culturing cells. The embodiments of the present invention can be used to culture different variety cells, such as eukaryotic and prokaryotic cells, particularly animal cells and/or mammalian cells. More particularly, anchorage-dependent animal cells.

The cell-cultivation apparatus of present invention includes a gas permeable chamber with at least one open end, a sealing member is detachably mounted at the at least one open end, and a growth substrate is disposed in the gas permeable chamber. The sealing element is a cap or a substrate holder, and the growth substrate is porous matrices in a disc-shape or a strip-shape. Below describing detailed parts of the cell-cultivation apparatus in different embodiments.

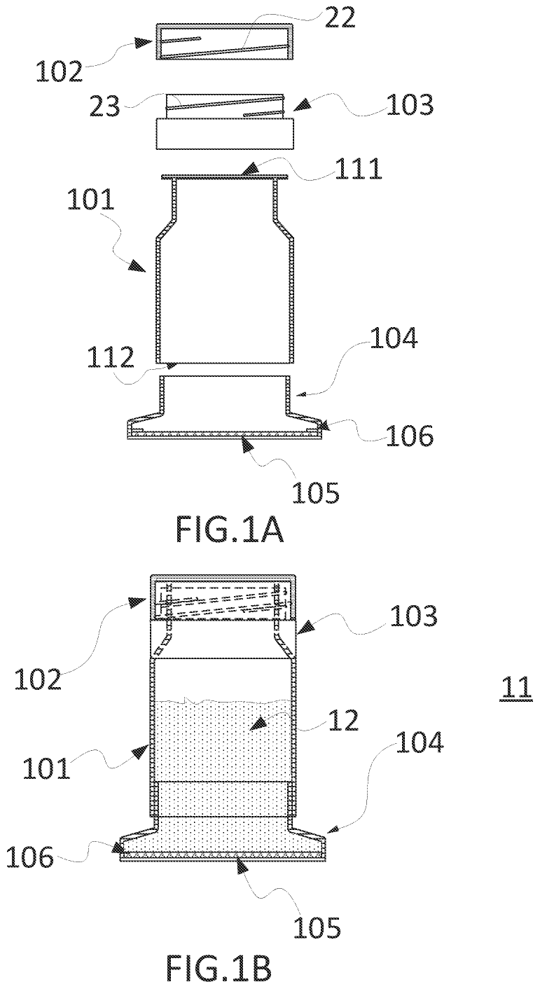

The apparatus for preparing and culturing cells shown in FIG. 1A, FIG. 1B show side views of a cell-cultivation apparatus of the first embodiment of the present invention with pre-assemble (FIG. 1A) and post-assemble (FIG. 1B) for ease of understanding. The cell-cultivation apparatus includes a gas permeable chamber 101 which is preferably a bottle shape chamber with a first open end 111 and a second open end 112. A sealing member may include a cap 102 detachably mounted at the first end 111. A growth substrate holder 104 and the growth substrate 105 are detachably mounted at the second end 112 of the gas permeable chamber 101. The growth substrate 105 is a porous sheet or disc that is fixed by a wedge ring 106 molded in the growth substrate holder 104. The growth substrate holder 104 is open at top for culture medium and gas transfer and exchange during cell culture. The gas permeable chamber 101 is preferably made by silicone rubber which can be semi-rigid under certain thickness from 0.5 mm to 2 mm, more preferably, from 0.5 to 1 mm, and it can be stand still without requiring extra supporting means. The gas permeable chamber 101 is closed by the cap 102. In order to secure the cap 102 to be air-tight with the gas permeable chamber 101, a rigid neck or a rigid tube 103 with external threads 23 is detachably mounted on the gas permeable chamber 101 in order to engage with the internal threads 22 of the cap 102. Silicone rubber has proven to be good materials for the gas exchange membrane. A silicone rubber can be manufactured economically in any desired shape by injection molding. Silicone rubber is available commercially in many thicknesses, shapes, and specific gas permeability. It has high tear resistance and good chemical resistance to the medium ordinarily used in cell culturing, and it is therefore also especially easy to handle. It is understood that the threads for engagement may be directly made on the open end of the gas permeable chamber 101 in case that silicone rubber is rigid enough for processing.

Referring to FIG. 1A and FIG. 1B again, the gas permeable chamber 101 may accommodate the culture medium 12, and the culture medium 12 in liquid phase may be enclosed within the gas permeable chamber 101 of the cell-cultivation apparatus 11 after post assembling. The culture medium 12 does not full in the gas permeable chamber 101 to leave a little space for gas. The gas may be in and out of the gas permeable chamber 101 due to gas permeability of the cell-cultivation apparatus 11.

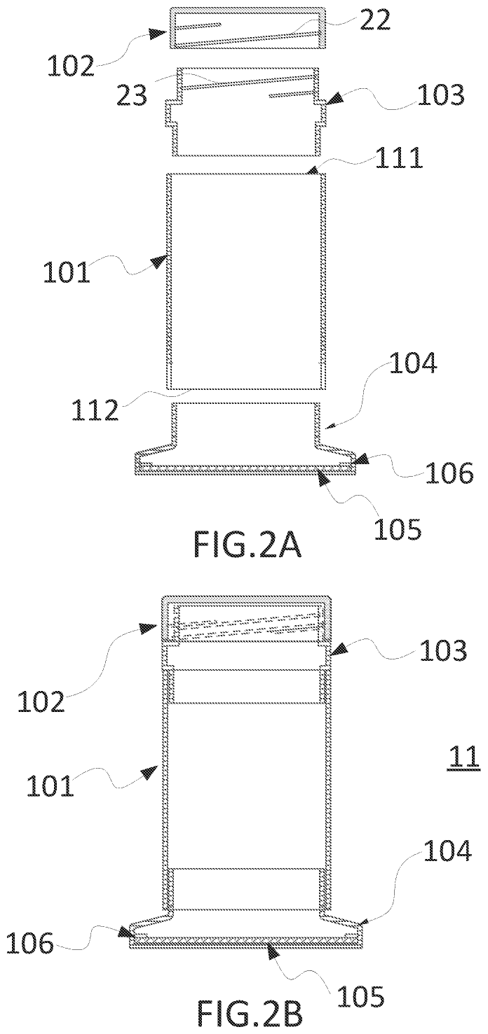

The apparatus for preparing and culturing cells shown in FIG. 2A and FIG. 2B show side views of a cell-cultivation apparatus of the second embodiment of the present invention with pre-assemble (FIG. 2A) and post-assemble (FIG. 2B) for ease of understanding. Compared to the first embodiment, in addition to one part with the thread 23 for engaging with the cap 22, the rigid tube 103 provides a narrow part to plug into the first end 111 of the gas permeable chamber 101 and a wide part to lean on the gas permeable chamber 101. Furthermore, the gas permeable chamber 101 is preferably made by silicone rubber with thickness from 0.5 to 2 mm, more preferably, from 0.5 to 1 mm, which can be semi-rigid under certain thickness and can be stand still without requiring extra supporting means.

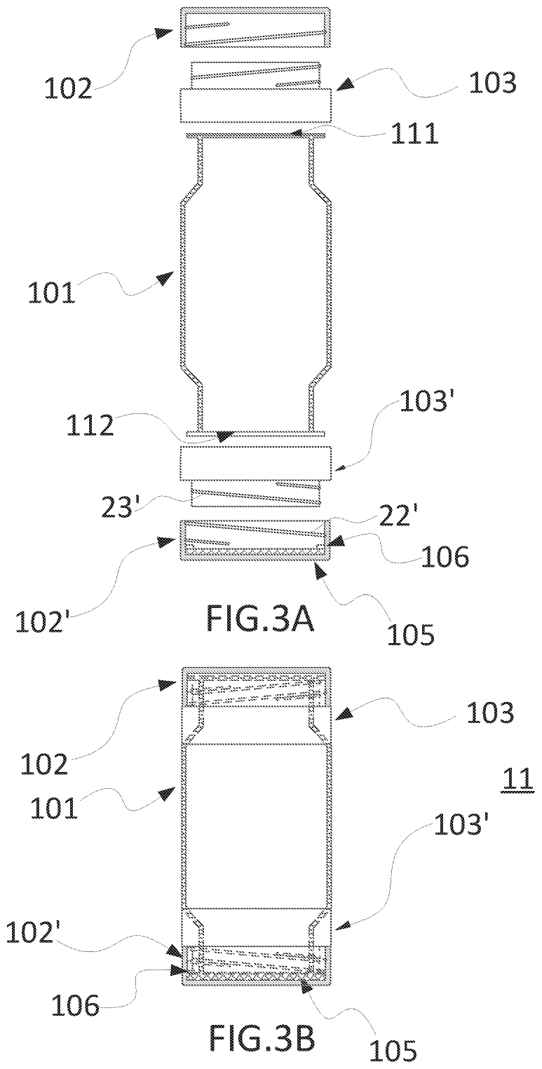

The apparatus for preparing and culturing cells shown in FIG. 3A, FIG. 3B shows a side view of a cell-cultivation apparatus of the third embodiment of the present invention with pre-assemble (FIG. 3A) and post-assemble (FIG. 3B) for ease of understanding. The cell-cultivating apparatus includes a gas permeable chamber 101 with a first open end 111 and a second open end 112, wherein a cap 102 with threads is detachably mounted in the first end, and another cap 102′ with threads 22′ and a growth substrate 105 are detachably mounted at the second end of the gas permeable chamber 101. The growth substrate 105 is a porous sheet or disc that is fixed by a wedge ring 106 molded in another cap 102′. In order to secure the caps 102, 102′ to be air tight, rigid necks 103, 103′ with external threads 23, 23′ are detachably mounted on the gas permeable chamber 101 both on top and bottom open ends in order to engage and secure with internal threads 22, 22′ of the caps 102, 102′.

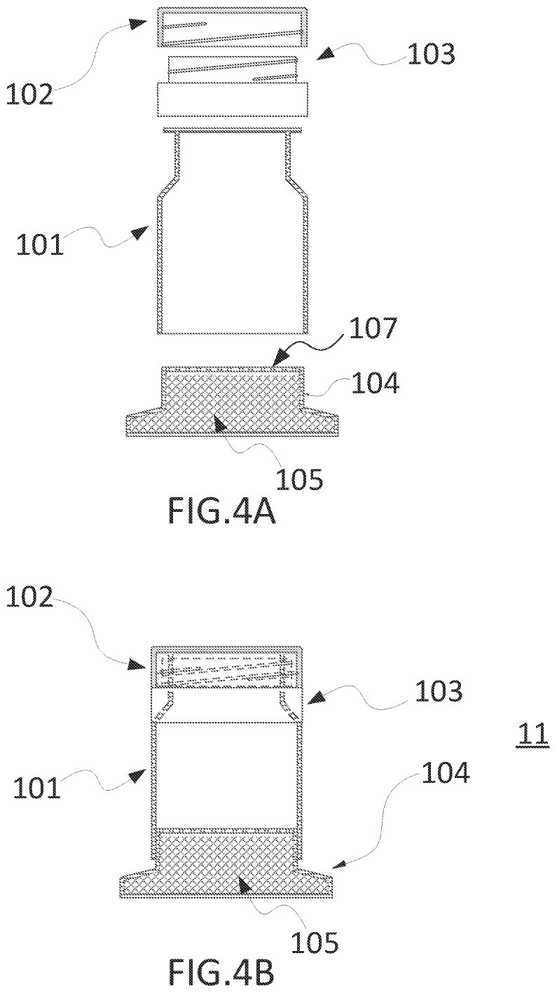

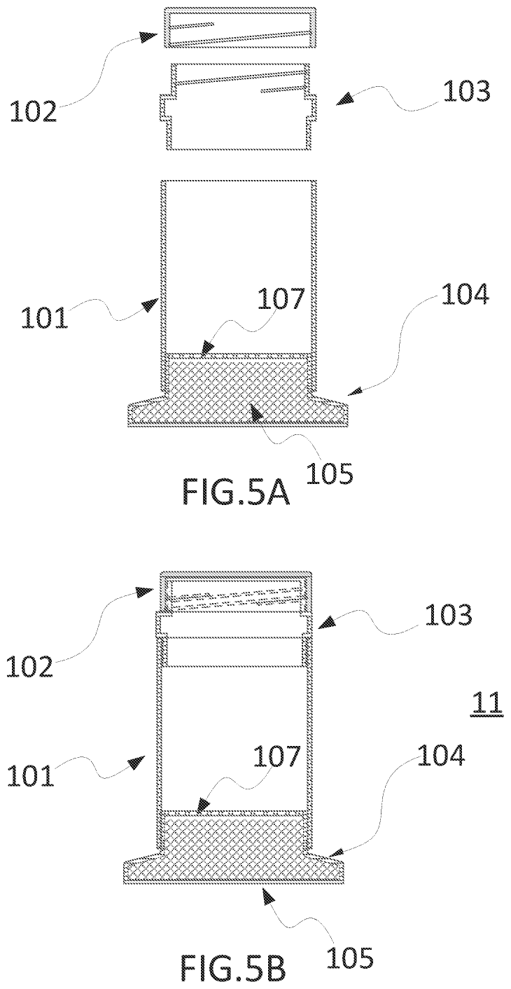

The apparatus for preparing and culturing cells shown in FIG. 4A, 4B and FIG. 5A, FIG. 5B show side views of a cell-cultivation apparatus of the fourth and fifth embodiments of the present invention with pre-assemble (FIG. 4A, FIG. 5A) and post-assemble (FIG. 4B, FIG. 5B) for ease of understanding. The cell-cultivation apparatus 11 includes a gas permeable chamber 101 with a first open end and a second open end, wherein a cap 102 is detachably mounted at the first end, and a growth substrate holder 104 and the growth substrate 105 is detachably mounted at the second end of the gas permeable chamber 101. The growth substrate 105 is a plurality of discs or strips of porous matrices, such as BioNOC II carriers (CESCO Bioengineering Co., Ltd., Taiwan), that are disposed in the growth substrate holder 104. The growth substrate holder 104 is a basket that has top openings 107 for transfer and exchange of culture medium and air during operation. The gas permeable chamber 101 is closed by a rigid cap 102 with threads. In order to secure the cap 102 to be air tight, a rigid neck or a rigid tube 103 with external threads is detachably mounted on the gas permeable chamber 101 in order to engage and secure with the cap 102.

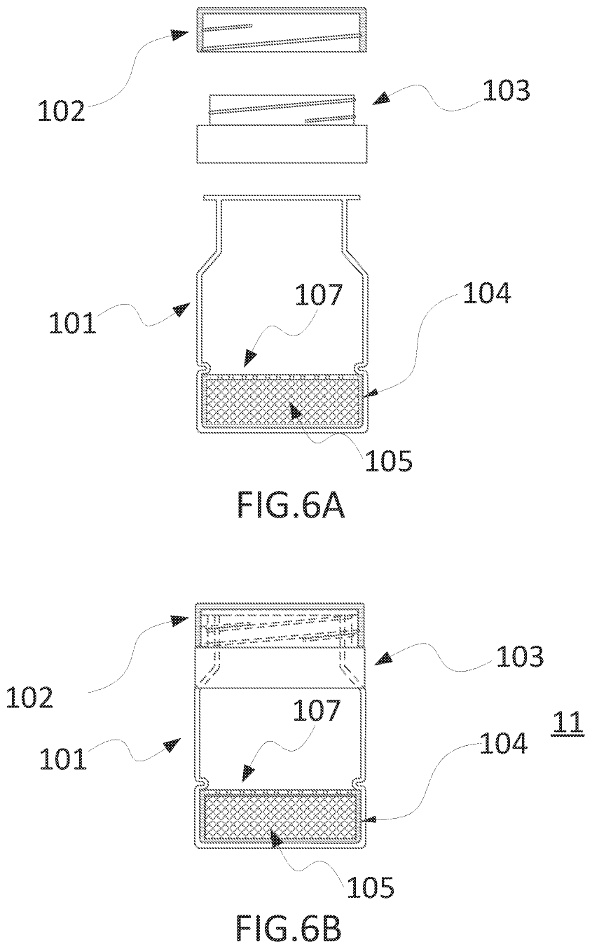

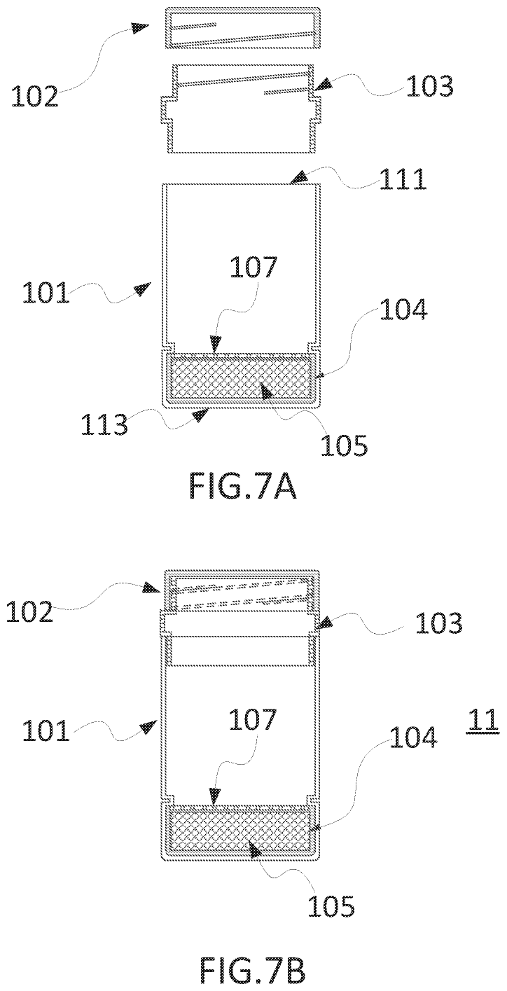

The apparatus for preparing and culturing cells in FIG. 6A, FIG. 6B and FIG. 7A, FIG. 7B show side views of a cell-cultivation apparatus of the sixth and seventh embodiments of the present invention with pre-assemble (FIG. 6A, FIG. 7A) and post-assemble (FIG. 6B, FIG. 7B) for ease of understanding. The cell-cultivation apparatus includes a gas permeable chamber 101 with an open end 111 and a close end 113, wherein a cap 102 is detachably mounted at the open end 111, and a growth substrate holder 104 and a growth substrate 105 is detachably mounted at the close end 113 of the gas permeable chamber 101. The growth substrate 105 is a plurality of discs or strips of porous matrices, such as BioNOC II carriers (CESCO Bioengineering Co., Ltd., Taiwan), that are disposed in the growth substrate holder 104. The growth substrate holder 104 is a basket that has top openings 107 for transfer and exchange of culture media and air during operation. The gas permeable chamber 101 is closed by a rigid cap 102. In order to secure the cap 102 to be air tight, a rigid neck or a rigid tube 103 with external threads is detachably mounted on the gas permeable chamber 101 in order to engage and secure with the cap 102. It's optional to use other designs to secure the cap such as flanges.

Accordingly, the gas permeable chamber of silicon rubber is beneficial for users to see through during cell cultivation. That is, the growth substrate and culture medium in the gas permeable chamber are visible for user to observe them from outside of the gas permeable chamber. It is also noted that the growth substrate may be deposited at a suitable position within the gas permeable chamber where it may be repeatedly submerged in or exposed out of the culture medium in the gas permeable chamber.

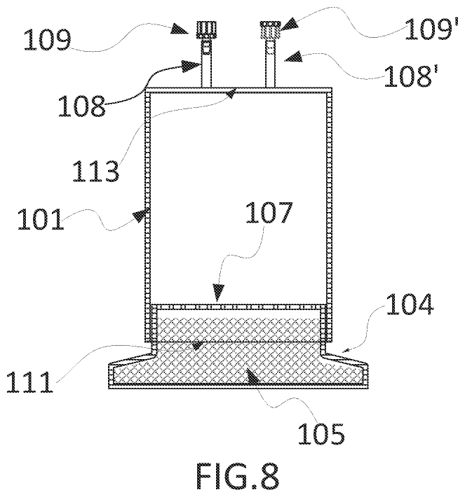

The apparatus for preparing and culturing cells shown in FIG. 8 shows a side view of a cell-cultivation apparatus of the eighth embodiment of the present invention, especially for close operation. The cell-cultivation apparatus includes a gas permeable chamber 101 with a close end 113 and an open end 111, wherein a plurality of openings with tubes 108, 108′ are detachably mounted at the close end 113 of the gas permeable chamber 101, and these openings with tubes 108, 108′ are close by fittings 109, 109′ such as luer fittings. A growth substrate holder 104 and a growth substrate 105 is detachably mounted at the open end 111 of the gas permeable chamber 101. The growth substrate 105 is a plurality of discs or strips of porous matrices, such as BioNOC II carriers (CESCO Bioengineering Co., Ltd., Taiwan), that are disposed in the growth substrate holder 104. The growth substrate 105 could also be a sheet of porous matrices. The growth substrate holder 104 is a basket that has top openings 107 for transfer and exchange of culture media and air during operation.

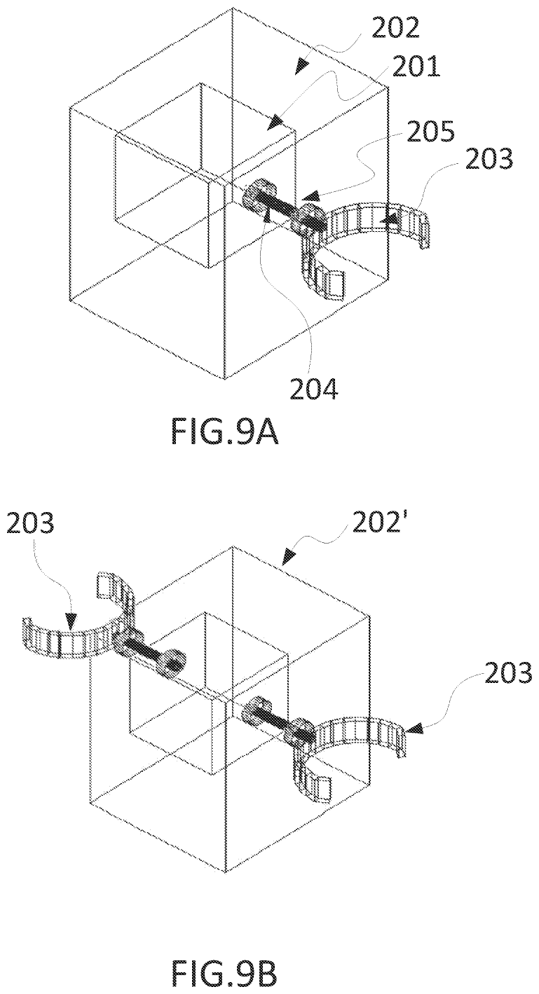

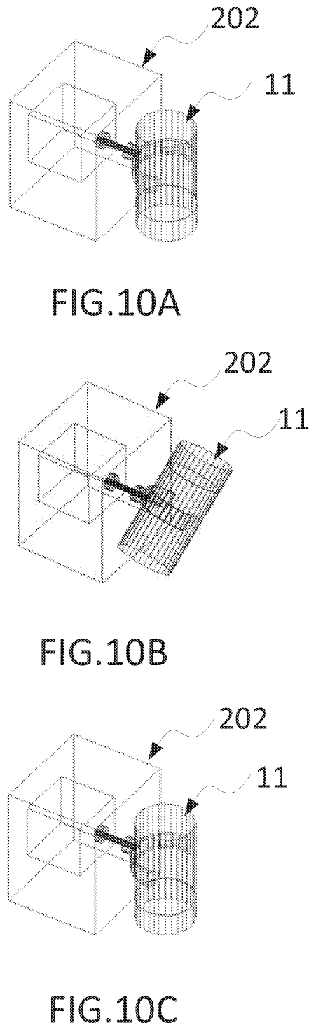

FIG. 9A and FIG. 9B show devices for rotating the cell cultivation apparatus for cell growth according to the present invention. The devices 202, 202′ respectively shown in FIG. 9A and FIG. 9B include at least one clamp or holder or platform 203 disposed to hold the cell-cultivation apparatus, and the clamp or the holder or the platform 203 is connected to a motor 201 through a shaft 204 and bearings 205, preferably two-way step motor. Shown from FIG. 10A, FIG. 10B to FIG. 10C, the motor of the device 202 may rotate slowly clockwise or counterclockwise until reach 180 degree of angle, and then stops and holds the cell cultivation apparatus 11 with an interval time. A timing controller (not shown) is used to control the interval time. Then the motor of the device 202 rotates again counterclockwise or clockwise slowly until reach 180 degree of angle, and then stops again and holds the cell cultivation apparatus with the interval time. The degree of angle is not necessary to reach 180 degree but to expose and submerge the growth substrate is the target that determines the minimum degree of angle for rotation.

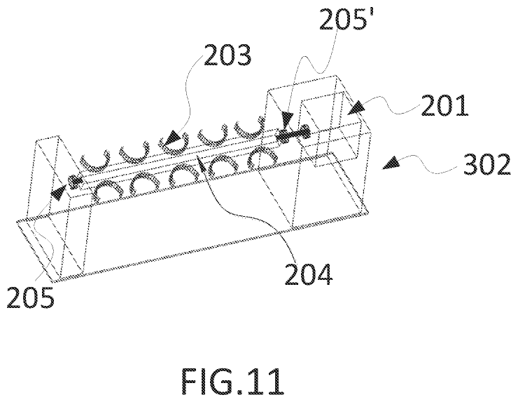

The apparatus for preparing and culturing cells in FIG. 11 shows the cell cultivation apparatus of the present invention where the device 302 includes a plurality of clamps or holders 203 is disposed to hold the cell-cultivation apparatus, the clamps or the holders 203 connected to a motor 201 through a shaft 204 and bearings 205, 205′, preferably two-way step motor. The motor 201 rotates slowly clockwise or counterclockwise until reach 180 degree of angle, and then stops and holds the cell cultivation apparatus with an interval time. A timing controller (not shown) is used to control the interval time. Then the motor 201 rotates again counterclockwise or clockwise slowly until reach 180 degree of angle, and then stops again and holds the cell cultivation apparatus with the interval time. The degree of angle is not necessary to reach 180 degree but to expose and submerge the growth substrate 105 (referring to FIG. 1A, and so on) is the target that determines the minimum degree of angle for rotation. The rotation speed is smaller than 10 rpm, or greater than 0 and smaller than or equal to 2 rpm, or preferably, greater than 0 and smaller than or equal to 1 rpm. The interval time at upright and bottom down position is about or less than 60 minutes, preferably, about 10 minutes. Accordingly, the device 302 executes a rotation process for cell cultivation apparatus, and the rotation process includes clockwise or counterclockwise rotating the cell cultivation apparatus and maintain the cell cultivation apparatus to be still with the interval time between two rotating steps.

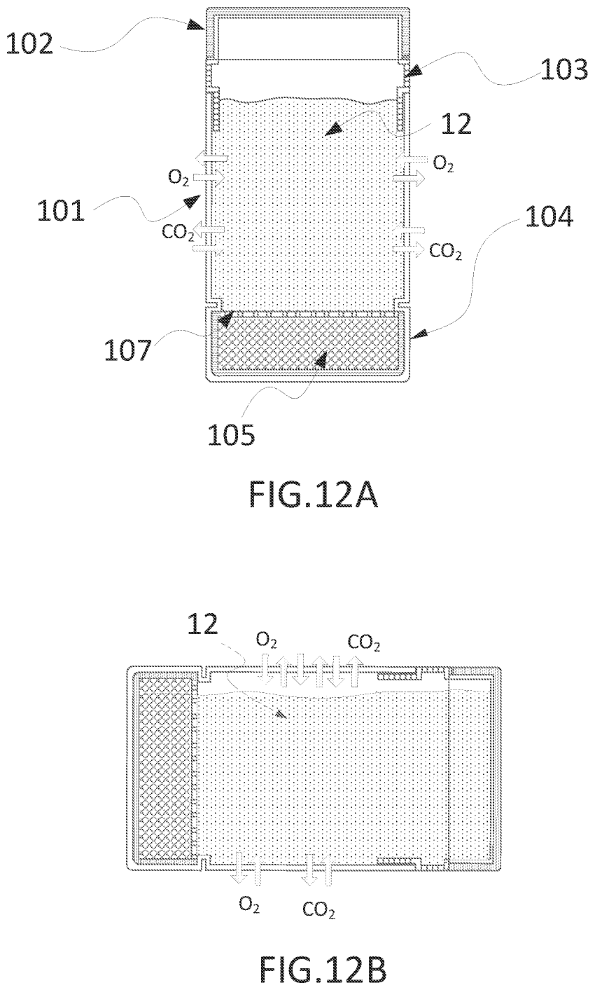

The apparatus for preparing and culturing cells shown in FIG. 12A, FIG. 12B and FIG. 12C shows the mechanism of a cell-cultivation apparatus of the present invention, as an example from all designs, where in the culture medium 12 is partially filled in the gas permeable chamber 101, the growth substrate 105 is at the bottom of the gas permeable chamber 101 at the beginning (for example) and is submerged in the culture medium 12. The amount of the filled culture medium 12 is judged by the capability to expose the cell growth substrate 105 completely when rotating the gas permeable chamber 101 and the growth substrate 105 is at top position.

After mounting the cell cultivating apparatus on the driving device, as disclosed in FIG. 10A and FIG. 12A, and start rotation, the gas permeable chamber 101 is then rotated clockwise or counterclockwise where in part of the gas permeable chamber 101 is exposed to gaseous phase and part of the gas permeable chamber 101 is submerged to culture medium 12 of liquid phase. The gas permeable chamber 101 keeps rotating clockwise or counterclockwise continuously (middle state shown in FIG. 12B) until the growth substrate 105 reach at the top position (shown in FIG. 12C) where the growth substrate 105 in the higher end of the gas permeable chamber 101 is thus emerge from the culture medium and exposed to the gaseous environment. Then rotating the gas permeable chamber 101 upside-down to submerge the substrate in the medium. The partial filled of culture medium 12 in the gas permeable chamber 101 can be used to eliminate the boundary effect of oxygen and carbon dioxide transfer by skimpily remove the liquid with gaseous head space by rotating a partial liquid filled gas permeable chamber 101. The partial filled of culture medium in the gas permeable chamber 101 can also be used to expose the growth substrate 105 to the gaseous phase and enhance gas transfer rate for both oxygen and carbon dioxide. Through this special design and mechanism, the oxygen and carbon dioxide transfer rate can be further accelerated, and a result of high-density cell culture could be reached, without requiring vigorous agitation and/or bubble sparging.

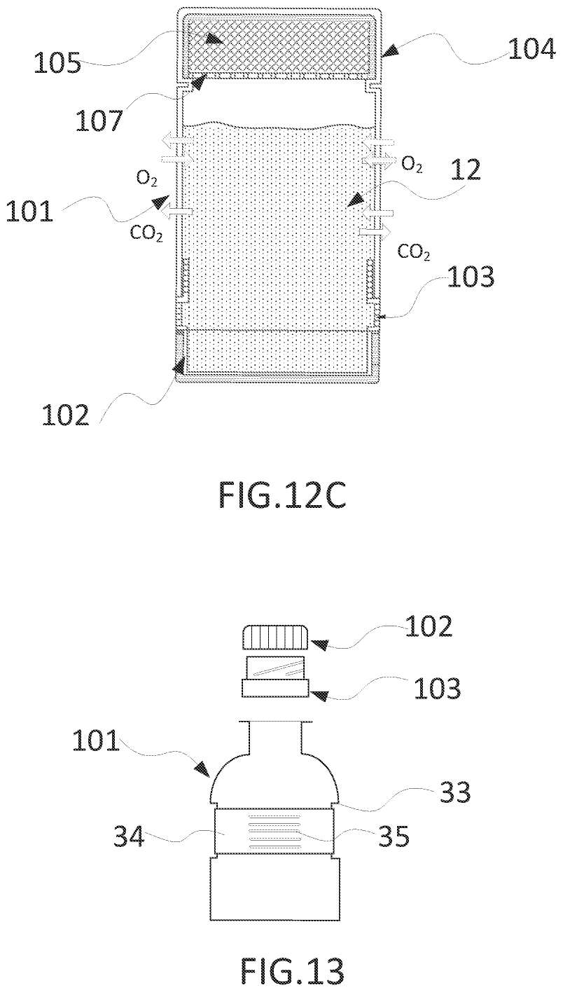

Shown in FIG. 13, in the ninth example, the gas permeable chamber 101 may provide a narrow portion 33 for fitting of the clamp of the device 202 (shown in FIG. 9A) for driving. An additional member 34 may be mounted onto the narrow portion 33 for supporting. The member 34 may be transparent without blocking visibility. There may be some marks 35 on the member 34 for liquid level aligning. Optionally, the marks 35 for liquid level aligning may be directly attached onto the suitable position of wall of the gas permeable chamber 101.

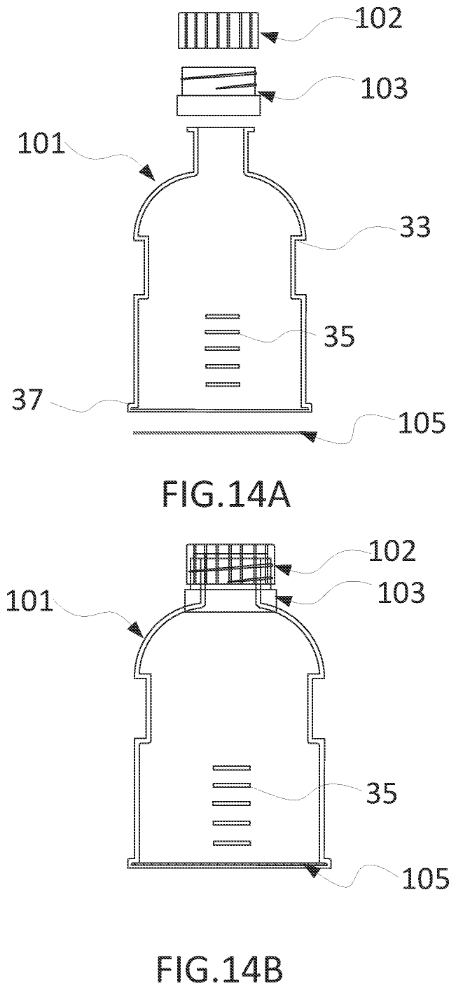

FIG. 14A and FIG. 14B show side views of a cell-cultivation apparatus of the tenth embodiment of present invention with pre-assemble and post-assemble. In addition to the narrow portion 33 in the nineth example, the gas permeable chamber 101 further includes a fixing portion 37 at the close end of the gas permeable chamber 101. Moreover, the marks 35 are directed formed on the wall of the gas permeable chamber 101 and the wall of the gas permeable chamber 101 is transparent enough for a user to see the level of culture medium in the gas permeable chamber 101. A sheet of the growth substrate 105 may be put into the gas permeable chamber 101 and mounted by the fixing portion 37. And the bottom close end of the gas permeable chamber 101 may be transparent enough for a user to see the sheet of the growth substrate 105.

The present invention is directed to a reliable, simple, inexpensive, disposable, sterile and efficient method for culturing cells and/or tissues and harvesting cellular products produced by cells cultured thereof. More specifically, the present invention provides a novel method for efficiently culturing any cells whether eukaryotic, prokaryotic, mammalian or animal wherein both oxygen and nutrients needed to ensure cell growth are readily available without causing damage to cells. Furthermore, the method of the present invention prevents or greatly reduces the metabolite waste accumulation, avoid introducing shear forces on growing cultures, and protect cells from direct exposure to gas, air bubbles and gases. Further still, the instant invention provides a method for an easier and more convenient means for producing and harvesting secreted cellular products such as proteins, antibodies from cell or tissue cultures.

The VERO cells, a common cell line for virus expression, were grown according to the instant invention. The first step was to open the cap on the gas permeable vessel (60 ml total volume) made by silicone rubber, where around 20 pieces of BioNOC II (occupy around 1.5 ml) are packed and fixed at the bottom end of the gas permeable vessel, then 1×10⁷ VERO cells were introduced with 50 ml culture medium into the vessel, the cap was closed and make sure it was air tight, make sure the carriers can be exposed to gaseous headspace after reversing the vessel, mounted the gas permeable vessel on the clamp set on the device, set up rotation rate as 0.3 rpm, top interval time is 10 seconds, and bottom interval time is 10 seconds, and started the culture. The device rotated the gas permeable vessel clockwise with 0.3 rpm it until reach 180 degree of angle until the gas permeable vessel was upside down and the carriers were exposed, then will hold 10 seconds, then the device rotated the gas permeable vessel counterclockwise with 0.3 rpm until reached 180 degree of angle until the gas permeable vessel stood upright and the carriers were submerged in the culture medium, another 10 seconds was hold at the position before starting another cycle. After 3 days culture, the cell density reached 3.891×10⁷ by estimating the cell number with sampled carriers. After 6 days culture, the cell density reaches 7.91×10⁷, and the carriers were filled with cells when observed under microscope.

The embodiments described above are merely illustrative of the technical spirit and features of the present invention, and are intended to enable those skilled in the art to understand the present invention and practice the present invention. The scope of the patent, that is, the equivalent changes or modifications made by the spirit of the present invention, should still be included in the scope of the patent of the present invention.

What is claimed is:

1. A cell-cultivation apparatus, comprising:
a gas permeable chamber with at least one open end to accommodate culture medium;
a sealing member detachably mounted at the at least one open end; and
a growth substrate disposed in the gas permeable chamber.

2. The cell-cultivation apparatus according to claim 1, wherein the sealing member comprises a cap and a rigid neck or a rigid tube, and the rigid neck or the rigid tube is detachably mounted at the at least one open end to engage with the cap.

3. The cell-cultivation apparatus according to claim 1, wherein the growth substrate is porous matrices in a disc-shape, in a sheet shape or a strip-shape.

4. The cell-cultivation apparatus according to claim 1, wherein the gas permeable chamber is with two the open ends.

5. The cell-cultivation apparatus according to claim 4, wherein two the sealing members detachably and respectively mounted at two the open ends, and one of two the sealing members includes a cap or a substrate holder disposed to fix the growth substrate.

6. The cell-cultivation apparatus according to claim 1, wherein the gas permeable chamber further comprises a close end.

7. The cell-cultivation apparatus according to claim 6, further comprising a basket disposed at the close end to fix the growth substrate.

8. The cell-cultivation apparatus according to claim 6, wherein the gas permeable chamber further comprises a fixing portion at the close end for fixing the growth substrate.

9. The cell-cultivation apparatus according to claim 6, further comprising an opening connected with a tube at the close end, wherein the tube is closed or opened by a fitting.

10. A device for rotating the cell-cultivation apparatus according to claim 1, comprising:
a motor;
a holder disposed to hold the cell-cultivation apparatus; and
a shaft connected between the motor and the holder to be driven by the motor to rotate the cell-cultivation apparatus.

11. The device according to claim 10, further comprising a timing controller connected to the motor to control a rotation process of the cell-cultivation apparatus.

12. A method for culturing cell with the cell-cultivation apparatus according to claim 1, comprising:
partially filling the culture medium into the gas permeable chamber to cover the growth substrate;
rotating the gas permeable chamber upside-down to expose the growth substrate from the culture medium; and
rotating the gas permeable chamber upside-down to submerge the growth substrate in the culture medium.

13. The method according to claim 12, further comprising maintaining the gas permeable chamber to be still with an interval time between the rotating steps.

14. The method according to claim 13, wherein the interval time is less than 60 minutes.

15. The method according to claim 12, wherein a rotation speed is greater than 0 and smaller than or equal to 2 rpm.

16. The method according to claim 12, wherein a rotation speed is smaller than 10 rpm.
